(19) United States
(12) Patent Application Publication
(10) Pub. No.: US 2022/0344046 A1
(43) Pub. Date: Oct. 27, 2022

(54) METHODS, SYSTEMS, APPARATUSES, AND DEVICES FOR FACILITATING MANAGING ADMINISTERING OF PSYCHEDELIC DRUGS TO USERS

(71) Applicant: New Pill Technologies, Inc., Brooklyn, NY (US)

(72) Inventor: Paul Chu, Brooklyn, NY (US)

(21) Appl. No.: 17/711,615

(22) Filed: Apr. 1, 2022

Related U.S. Application Data
(60) Provisional application No. 63/179,812, filed on Apr. 26, 2021.

(51) Int. Cl. G16H 50/20; G16H 20/13; G16H 20/70; G16H 10/60; G16H 40/67

(52) U.S. Cl. CPC G16H 50/20; G16H 20/13; G16H 20/70; G16H 10/60; G16H 40/67

(57) ABSTRACT

Disclosed herein is a device for facilitating managing administering of psychedelic drugs to users, in accordance with some embodiments. Accordingly, the apparatus comprises a dispenser unit, a communication device, a processing device, and a storage device. Further, the dispenser unit dispenses a dosage of a type of psychedelic drug. Further, the communication device receives user data. Further, the processing device analyzes the user data, determines mental disorder of the users, analyzes clinical data and research data using machine learning models, generates treatment plans for treating the mental disorder of the one or more users, determines the dosage of the type of psychedelic drug, and generates commands for the dispenser unit. Further, the dispensing of the dosage of the type of psychedelic drug is based on the commands. Further, the storage device retrieves the clinical data, retrieves the research data, and stores the machine learning models and the user data.

### FIELD OF THE INVENTION

The present disclosure relates to the field of article dispensing. More specifically, the present disclosure relates to methods, systems, apparatuses, and devices for facilitating managing administering of psychedelic drugs to users.

### BACKGROUND OF THE INVENTION

The field of article dispensing is technologically important to several industries, business organizations, and/or individuals.

Psychedelics are a subset of hallucinogenic drugs whose primary effect is to trigger nonordinary states of consciousness (known as psychedelic experiences or “trips”) via serotonin 2A receptor agonist. Nowadays, with the increasing stress and anxiety, the number of people suffering from mental disorders such as opioid addiction, alcohol addiction, smoking addiction, post-traumatic stress disorder, and clinical depression is continuously increasing. People suffering from mental disorders often require a proper therapeutic healing plan to overcome the mental disorder.

Therefore, there is a need for improved methods, systems, apparatuses, and devices for facilitating managing administering of psychedelic drugs to users that may overcome one or more of the above-mentioned problems and/or limitations.

### SUMMARY OF THE INVENTION

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

Disclosed herein is an apparatus for facilitating managing administering of psychedelic drugs to users, in accordance with some embodiments. Accordingly, the apparatus may include a dispenser unit, a communication device, a processing device, and a storage device. Further, the dispenser unit may be configured for dispensing a dosage of one or more types of one or more psychedelic drugs. Further, the one or more types of the one or more psychedelic drugs may be stored in the dispenser unit. Further, the communication device may be disposed on the dispenser unit. Further, the communication device may be configured for receiving one or more user data associated with one or more users from one or more input devices. Further, the processing device may be disposed on the dispenser unit. Further, the processing device may be communicatively coupled with the communication device. Further, the processing device may be configured for analyzing the one or more user data. Further, the processing device may be configured for determining one or more mental disorders of the one or more users based on the analyzing of the one or more user data. Further, the processing device may be configured for analyzing one or more clinical data and one or more research data using one or more machine learning models. Further, the processing device may be configured for generating one or more treatment plans for treating the one or more mental disorders of the one or more users based on the analyzing of the one or more user data and the analyzing of the one or more clinical data and the one or more research data. Further, the one or more treatment plans may be personalized to the one or more users. Further, the processing device may be configured for determining the dosage of the one or more types of the one or more psychedelic drugs based on the one or more treatment plans. Further, the processing device may be configured for generating one or more commands for the dispenser unit based on the determining of the dosage of the one or more types of the one or more psychedelic drugs. Further, the dispenser unit may be communicatively coupled with the processing device. Further, the dispensing of the dosage of the one or more types of the one or more psychedelic drugs may be based on the one or more commands. Further, the storage device may be disposed on the dispenser unit. Further, the storage device may be communicatively coupled with the processing device. Further, the storage device may be configured for retrieving the one or more clinical data associated with one or more clinical studies on the one or more mental disorders based on the determining of the one or more mental disorders. Further, the storage device may be configured for retrieving the one or more research data associated with a treatment of the one or more mental disorders based on the determining of the one or more mental disorders. Further, the storage device may be configured for storing the one or more machine learning models and the one or more user data.

Further disclosed herein is an apparatus for facilitating managing administering of psychedelic drugs to users, in accordance with some embodiments. Accordingly, the apparatus may include a dispenser unit, a communication device, a processing device, and a storage device. Further, the dispenser unit may be configured for dispensing a dosage of one or more types of one or more psychedelic drugs. Further, the one or more types of the one or more psychedelic drugs may be stored in the dispenser unit. Further, the communication device may be disposed on the dispenser unit. Further, the communication device may be configured for receiving one or more user data associated with one or more users from one or more input devices. Further, the communication device may be configured for transmitting one or more therapist data to one or more output devices. Further, the processing device may be disposed on the dispenser unit. Further, the processing device may be communicatively coupled with the communication device. Further, the processing device may be configured for analyzing the one or more user data. Further, the processing device may be configured for determining one or more mental disorders of the one or more users based on the analyzing of the one or more user data. Further, the processing device may be configured for analyzing one or more clinical data and one or more research data using one or more machine learning models. Further, the processing device may be configured for generating one or more treatment plans for treating the one or more mental disorders of the one or more users based on the analyzing of the one or more user data and the analyzing of the one or more clinical data and the one or more research data. Further, the one or more treatment plans may be personalized to the one or more users. Further, the processing device may be configured for determining the dosage of the one or more types of the one or more psychedelic drugs based on the one or more treatment plans. Further, the processing device may be configured for generating one or more commands for the dispenser unit based on the determining of the dosage of the one or more types of the one or more psychedelic drugs. Further, the dispenser unit may be communicatively coupled with the processing device. Further, the dispensing of the dosage of the one or more types of the one or more psychedelic drugs may be based on the one or more commands. Further, the processing device may be configured for identifying one or more therapists for the one or more users based on the one or more treatment plans. Further, the one or more therapists may be personalized to the one or more users. Further, the storage device may be disposed on the dispenser unit. Further, the storage device may be communicatively coupled with the processing device. Further, the storage device may be configured for retrieving the one or more clinical data associated with one or more clinical studies on the one or more mental disorders based on the determining of the one or more mental disorders. Further, the storage device may be configured for retrieving the one or more research data associated with a treatment of the one or more mental disorders based on the determining of the one or more mental disorders. Further, the storage device may be configured for storing the one or more machine learning models and the one or more user data. Further, the storage device may be configured for retrieving the one or more therapist data associated with the one or more therapists based on the identifying of the one or more therapists.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is a side view of an apparatus for facilitating managing administering of psychedelic drugs to users, in accordance with some embodiments.

FIG. 2 is a side view of the apparatus, in accordance with some embodiments.

FIG. 3 is a side view of the apparatus, in accordance with some embodiments.

FIG. 4 is a side view of the apparatus, in accordance with some embodiments.

FIG. 5 is a side view of the apparatus, in accordance with some embodiments.

FIG. 6 is a side view of the apparatus, in accordance with some embodiments.

FIG. 7 is a side view of the apparatus, in accordance with some embodiments.

FIG. 8 is a side view of an apparatus for facilitating managing administering of psychedelic drugs to users, in accordance with some embodiments.

FIG. 9 is a side view of the apparatus, in accordance with some embodiments.

FIG. 10 is a block diagram of a system for facilitating administering psychedelic dosing and regimen therapy tracking, in accordance with some embodiments.

FIG. 11 is a flow chart of a method for facilitating administering psychedelic dosing and regimen therapy tracking, in accordance with some embodiments.

FIG. 12 is a schematic of a system for facilitating administering psychedelic dosing and regimen therapy tracking, in accordance with some embodiments.

FIG. 13 is an illustration of an online platform consistent with various embodiments of the present disclosure.

FIG. 14 is a block diagram of a computing device for implementing the methods disclosed herein, in accordance with some embodiments.

### DETAIL DESCRIPTIONS OF THE INVENTION

All illustrations of the drawings are for the purpose of describing selected versions of the present invention and are not intended to limit the scope of the present invention.

Although the invention has been explained in relation to its preferred embodiment, it is to be understood that many other possible modifications and variations can be made without departing from the spirit and scope of the invention.

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of systems, methods, apparatuses, and devices for facilitating managing administering of psychedelic drugs to users, embodiments of the present disclosure are not limited to use only in this context.

In general, the method disclosed herein may be performed by one or more computing devices. For example, in some embodiments, the method may be performed by a server computer in communication with one or more client devices over a communication network such as, for example, the Internet. In some other embodiments, the method may be performed by one or more of at least one server computer, at least one client device, at least one network device, at least one sensor and at least one actuator. Examples of the one or more client devices and/or the server computer may include, a desktop computer, a laptop computer, a tablet computer, a personal digital assistant, a portable electronic device, a wearable computer, a smart phone, an Internet of Things (IoT) device, a smart electrical appliance, a video game console, a rack server, a super-computer, a mainframe computer, mini-computer, micro-computer, a storage server, an application server (e.g. a mail server, a web server, a real-time communication server, an FTP server, a virtual server, a proxy server, a DNS server etc.), a quantum computer, and so on. Further, one or more client devices and/or the server computer may be configured for executing a software application such as, for example, but not limited to, an operating system (e.g. Windows, Mac OS, Unix, Linux, Android, etc.) in order to provide a user interface (e.g. GUI, touch-screen based interface, voice based interface, gesture based interface etc.) for use by the one or more users and/or a network interface for communicating with other devices over a communication network. Accordingly, the server computer may include a processing device configured for performing data processing tasks such as, for example, but not limited to, analyzing, identifying, determining, generating, transforming, calculating, computing, compressing, decompressing, encrypting, decrypting, scrambling, splitting, merging, interpolating, extrapolating, redacting, anonymizing, encoding and decoding. Further, the server computer may include a communication device configured for communicating with one or more external devices. The one or more external devices may include, for example, but are not limited to, a client device, a third party database, public database, a private database and so on. Further, the communication device may be configured for communicating with the one or more external devices over one or more communication channels. Further, the one or more communication channels may include a wireless communication channel and/or a wired communication channel. Accordingly, the communication device may be configured for performing one or more of transmitting and receiving of information in electronic form. Further, the server computer may include a storage device configured for performing data storage and/or data retrieval operations. In general, the storage device may be configured for providing reliable storage of digital information. Accordingly, in some embodiments, the storage device may be based on technologies such as, but not limited to, data compression, data backup, data redundancy, deduplication, error correction, data finger-printing, role based access control, and so on.

Further, one or more steps of the method disclosed herein may be initiated, maintained, controlled and/or terminated based on a control input received from one or more devices operated by one or more users such as, for example, but not limited to, an end user, an admin, a service provider, a service consumer, an agent, a broker and a representative thereof. Further, the user as defined herein may refer to a human, an animal or an artificially intelligent being in any state of existence, unless stated otherwise, elsewhere in the present disclosure. Further, in some embodiments, the one or more users may be required to successfully perform authentication in order for the control input to be effective. In general, a user of the one or more users may perform authentication based on the possession of a secret human readable secret data (e.g. username, password, passphrase, PIN, secret question, secret answer etc.) and/or possession of a machine readable secret data (e.g. encryption key, decryption key, bar codes, etc.) and/or or possession of one or more embodied characteristics unique to the user (e.g. biometric variables such as, but not limited to, fingerprint, palm-print, voice characteristics, behavioral characteristics, facial features, iris pattern, heart rate variability, evoked potentials, brain waves, and so on) and/or possession of a unique device (e.g. a device with a unique physical and/or chemical and/or biological characteristic, a hardware device with a unique serial number, a network device with a unique IP/MAC address, a telephone with a unique phone number, a smartcard with an authentication token stored thereupon, etc.). Accordingly, the one or more steps of the method may include communicating (e.g. transmitting and/or receiving) with one or more sensor devices and/or one or more actuators in order to perform authentication. For example, the one or more steps may include receiving, using the communication device, the secret human readable data from an input device such as, for example, a keyboard, a keypad, a touch-screen, a microphone, a camera and so on. Likewise, the one or more steps may include receiving, using the communication device, the one or more embodied characteristics from one or more biometric sensors.

Further, one or more steps of the method may be automatically initiated, maintained and/or terminated based on one or more predefined conditions. In an instance, the one or more predefined conditions may be based on one or more contextual variables. In general, the one or more contextual variables may represent a condition relevant to the performance of the one or more steps of the method. The one or more contextual variables may include, for example, but are not limited to, location, time, identity of a user associated with a device (e.g. the server computer, a client device etc.) corresponding to the performance of the one or more steps, environmental variables (e.g. temperature, humidity, pressure, wind speed, lighting, sound, etc.) associated with a device corresponding to the performance of the one or more steps, physical state and/or physiological state and/or psychological state of the user, physical state (e.g. motion, direction of motion, orientation, speed, velocity, acceleration, trajectory, etc.) of the device corresponding to the performance of the one or more steps and/or semantic content of data associated with the one or more users. Accordingly, the one or more steps may include communicating with one or more sensors and/or one or more actuators associated with the one or more contextual variables. For example, the one or more sensors may include, but are not limited to, a timing device (e.g. a real-time clock), a location sensor (e.g. a GPS receiver, a GLONASS receiver, an indoor location sensor etc.), a biometric sensor (e.g. a fingerprint sensor), an environmental variable sensor (e.g. temperature sensor, humidity sensor, pressure sensor, etc.) and a device state sensor (e.g. a power sensor, a voltage/current sensor, a switch-state sensor, a usage sensor, etc. associated with the device corresponding to performance of the or more steps).

Further, the one or more steps of the method may be performed one or more number of times. Additionally, the one or more steps may be performed in any order other than as exemplarily disclosed herein, unless explicitly stated otherwise, elsewhere in the present disclosure. Further, two or more steps of the one or more steps may, in some embodiments, be simultaneously performed, at least in part. Further, in some embodiments, there may be one or more time gaps between performance of any two steps of the one or more steps.

Further, in some embodiments, the one or more predefined conditions may be specified by the one or more users. Accordingly, the one or more steps may include receiving, using the communication device, the one or more predefined conditions from one or more and devices operated by the one or more users. Further, the one or more predefined conditions may be stored in the storage device. Alternatively, and/or additionally, in some embodiments, the one or more predefined conditions may be automatically determined, using the processing device, based on historical data corresponding to performance of the one or more steps. For example, the historical data may be collected, using the storage device, from a plurality of instances of performance of the method. Such historical data may include performance actions (e.g. initiating, maintaining, interrupting, terminating, etc.) of the one or more steps and/or the one or more contextual variables associated therewith. Further, machine learning may be performed on the historical data in order to determine the one or more predefined conditions. For instance, machine learning on the historical data may determine a correlation between one or more contextual variables and performance of the one or more steps of the method. Accordingly, the one or more predefined conditions may be generated, using the processing device, based on the correlation.

Further, one or more steps of the method may be performed at one or more spatial locations. For instance, the method may be performed by a plurality of devices interconnected through a communication network. Accordingly, in an example, one or more steps of the method may be performed by a server computer. Similarly, one or more steps of the method may be performed by a client computer. Likewise, one or more steps of the method may be performed by an intermediate entity such as, for example, a proxy server. For instance, one or more steps of the method may be performed in a distributed fashion across the plurality of devices in order to meet one or more objectives. For example, one objective may be to provide load balancing between two or more devices. Another objective may be to restrict a location of one or more of an input data, an output data and any intermediate data therebetween corresponding to one or more steps of the method. For example, in a client-server environment, sensitive data corresponding to a user may not be allowed to be transmitted to the server computer. Accordingly, one or more steps of the method operating on the sensitive data and/or a derivative thereof may be performed at the client device.

Overview:

The present disclosure describes methods, systems, and apparatuses for facilitating managing administering of psychedelic drugs to users. Further, the disclosed system may be configured for unique individualized dosing, delivery, and tracking of psychedelic, cannabis formulations. Further, the disclosed system may include at-home apparatus with user security validated dispenser (or dispenser) for dispensing liquid and solid bioactive psychedelic, Phytocannabinoid (Psilocybin, LSD, MDNA, DMT with cannabis THC, CBD, CBN, delta 8 compound formulation). Further, the dispenser may auto-replenish from producer to end-user.

Further, the disclosed system uses clinical data with aggregate research datasets to power the precise dosing regimen for treatment of mental disorders such as opioid addiction, alcohol addiction, smoking addiction, post-traumatic stress disorder, clinical depression, anxiety disorder, dementia, then captures the result for a full loop treatment outcome. Further, the disclosed systems may be associated with an artificial intelligence application based on a machine learning (ML) algorithm. Further, the artificial intelligence application may include an MLdriven wellness tracking software platform that draws from world research on psychedelic, cannabis research & treatment databases with clinical studies, university research, and published formulation treatment plan. It is then individually tailored, and dispensed from the apparatus authenticated from the licensed compound formulation. Further, the disclosed system (or remote Telehealth App, an exemplary embodiment of the disclosed herein) may be configured for patient screening, preparing individualized treatment plans, identifying trained therapists to fit patient treatment profile with positive treatment outcome environments, while incorporating, assisted AR (Augment Reality) and VR (Virtual Reality), in addition to the App's function to guide, track dosing, optimize effective healing dosing, therapy settings and monitor real-time.

In further embodiments, a method associated with the standard operating procedure for caring for a patient is disclosed. Further, the method may facilitate providing care to the patient both in-person and virtual. Further, the method may include self-act by the patient to provide full consent to participate. Further, the patient may provide full consent to participate in the treatment, with or without a physician, psychiatrist referral. Further, the method may include screening and psychiatric assessment. Further, the screening and psychiatric assessment may include a preliminary review of the patient's medical history and in-person or virtual consult with clinical staff associated with the disclosed system and machine learning protocol.

Further, the method may include providing treatment options. With or without psychiatric or doctor's approval, the method may include providing an additional in-person review to align the patient's needs and health to one of the psychedelic treatments and set intentions with targeted goals for their treatment(s).

Further, the method may include providing doses via guided dispensing and real-time health tracking. Further, the patient may arrive at a clinic for their individualized treatment with dosing that lasts between 4-8 hours (depending on dose). Further, the patient may be guided, and if at home, virtual guidance doses and access to real-time staff to assist in their session and ease the experience, ensuring treatment goals are achieved. Further, safety and comfort are maintained at all times and in real-time.

Further, the method may include post-treatment integration & outcome info capture. Further, at the end of the session, a licensed therapist may provide additional therapy in person or via the Telehealth app to conclude the session and ensure the patient is aware, safe, alert, and comfortably reintegrated post-treatment.

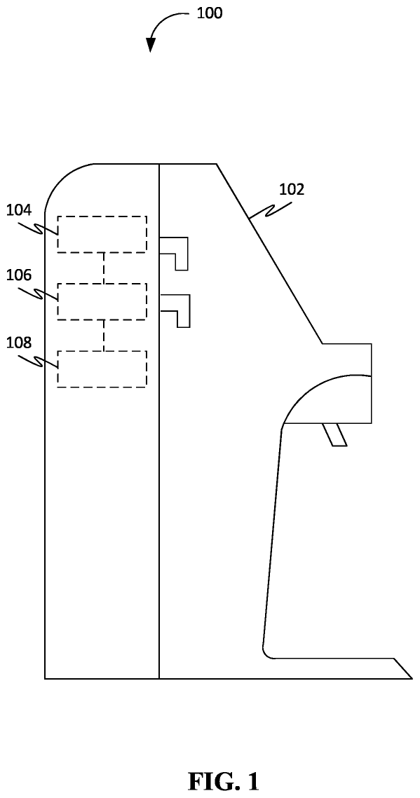

FIG. 1 is a side view of an apparatus 100 for facilitating managing administering of psychedelic drugs to users, in accordance with some embodiments. Further, the apparatus 100 may include a dispenser unit 102, a communication device 108, a processing device 106, and a storage device 104.

Further, the dispenser unit 102 may be configured for dispensing a dosage of one or more types of one or more psychedelic drugs. Further, the one or more types of the one or more psychedelic drugs may be stored in the dispenser unit 102. Further, the dispenser unit 102 may include an article dispenser. Further, the article dispenser may be command actuated. Further, the dispensing is based on a command. Further, the one or more types may include a solid, a liquid, a gaseous, etc. Further, the one or more psychedelic drugs may include Psilocybin, PCP, LSD, MDNA, DMT, peyote, mescaline, cannabis, etc. Further, the one or more psychedelic drugs may be alkaloids, polysaccharides, terpenoids, steroids, phenolics, etc.

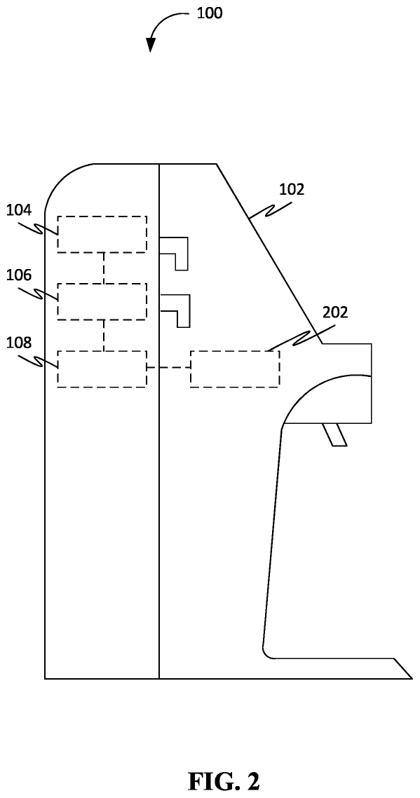

Further, the communication device 108 may be disposed on the dispenser unit 102. Further, the communication device 108 may be configured for receiving one or more user data associated with one or more users from one or more input devices 202, as shown in FIG. 2. Further, the communication device 108 may include a communication interface. Further, the one or more users may include individuals, patients, clients, etc. Further, the one or more input devices 202 may include the at least one client device. Further, the one or more user data may include a request, personal data and medical data.

Further, the processing device 106 may be disposed on the dispenser unit 102. Further, the processing device 106 may include a processing unit. Further, the processing device 106 may be communicatively coupled with the communication device 108. Further, the processing device 106 may be configured for analyzing the one or more user data. Further, the processing device 106 may be configured for determining one or more mental disorders of the one or more users based on the analyzing of the one or more user data. Further, the one or more mental disorders may include opioid addiction, alcohol addiction, smoking addiction, post-traumatic stress disorder, clinical depression, anxiety disorder, dementia, etc. Further, the processing device 106 may be configured for analyzing one or more clinical data and one or more research data using one or more machine learning models. Further, the one or more machine learning models may be trained to identify best suited psychedelic drugs and a therapy regimen of the psychedelic drugs for treating the one or more mental disorders of users. Further, the processing device 106 may be configured for generating one or more treatment plans for treating the one or more mental disorders of the one or more users based on the analyzing of the one or more user data and the analyzing of the one or more clinical data and the one or more research data. Further, the one or more treatment plans may be personalized to the one or more users. Further, the processing device 106 may be configured for determining the dosage of the one or more types of the one or more psychedelic drugs based on the one or more treatment plans. Further, the processing device 106 may be configured for generating one or more commands for the dispenser unit 102 based on the determining of the dosage of the one or more types of the one or more psychedelic drugs. Further, the dispenser unit 102 may be communicatively coupled with the processing device 106. Further, the dispensing of the dosage of the one or more types of the one or more psychedelic drugs may be based on the one or more commands.

Further, the storage device 104 may be disposed on the dispenser unit 102. Further, the storage device 104 may be communicatively coupled with the processing device 106. Further, the storage device 104 may be configured for retrieving the one or more clinical data associated with one or more clinical studies on the one or more mental disorders based on the determining of the one or more mental disorders. Further, the one or more clinical data may include scientific data, medical data, psychedelic treatment data resulting from the one or more clinical studies performed on individuals suffering from the one or more mental disorders. Further, the storage device 104 may be configured for retrieving the one or more research data associated with a treatment of the one or more mental disorders based on the determining of the one or more mental disorders. Further, the one or more research data may include any research papers, research articles, etc published on one or more of a formulation, a method of delivery, and a regimen of the one or more psychedelic drugs used for the treatment of the one or more mental disorders. Further, the storage device 104 may be configured for storing the one or more machine learning models and the one or more user data.

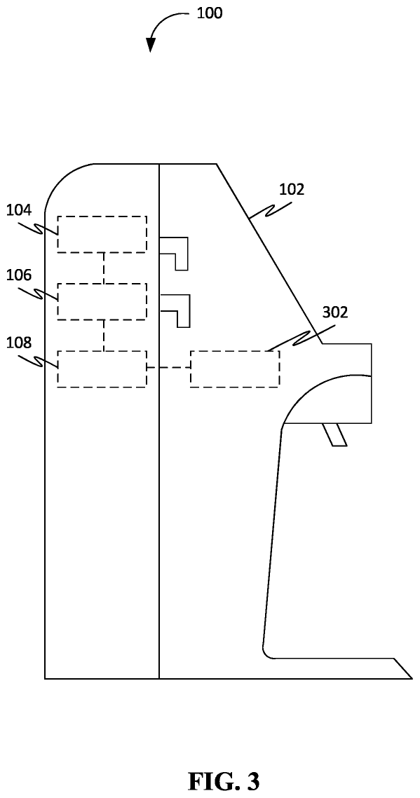

Further, in some embodiments, the processing device 106 may be configured for identifying one or more therapists for the one or more users based on the one or more treatment plans. Further, the one or more therapists may be personalized to the one or more users. Further, the storage device 104 may be configured for retrieving one or more therapist data associated with the one or more therapists based on the identifying of the one or more therapists. Further, the one or more therapist data may include personal details, pricing, credentials, contact information, etc. Further, the communication device 108 may be configured for transmitting the one or more therapist data to one or more output devices 302, as shown in FIG. 3. Further, the one or more output devices 302 may include the at least one client device.

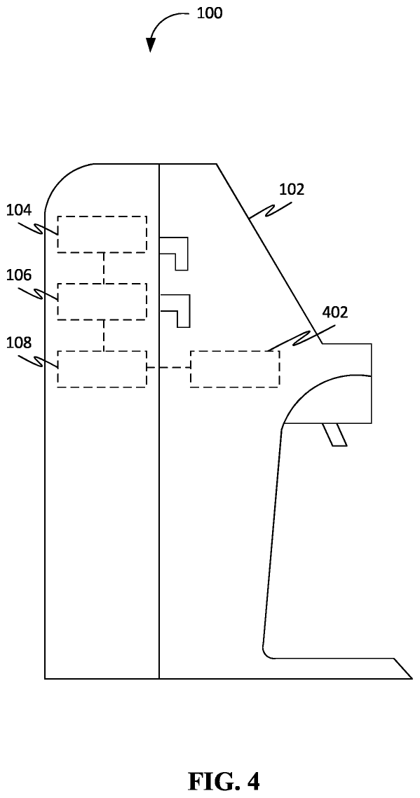

Further, in some embodiments, the communication device 108 may be configured for receiving one or more sensor data associated with the one or more users from one or more sensors 404, as shown in FIG. 4. Further, the one or more sensors 404 may be configured for generating the one or more sensor data based on detecting one or more of a physical parameter, a physiological parameter, and a biological parameter of the one or more users. Further, in an instance, the detecting of one or more of the physical parameter, the physiological parameter, and the biological parameter of the one or more users may be done in real time. Further, the processing device 106 may be configured for analyzing the one or more sensor data. Further, the generating of the one or more treatment plans may be based on the analyzing of the one or more sensor data. Further, in an instance, the generating of the one or more treatment plans may be done in real time. Further, the physical parameter may include a weight, etc. Further, the physiological parameter may include body temperature, ECG (heart rate), pulse rate, blood pressure, respiration rate, etc. Further, the biological parameter may include a level of one or more hormones, a level of one or more drugs, etc.

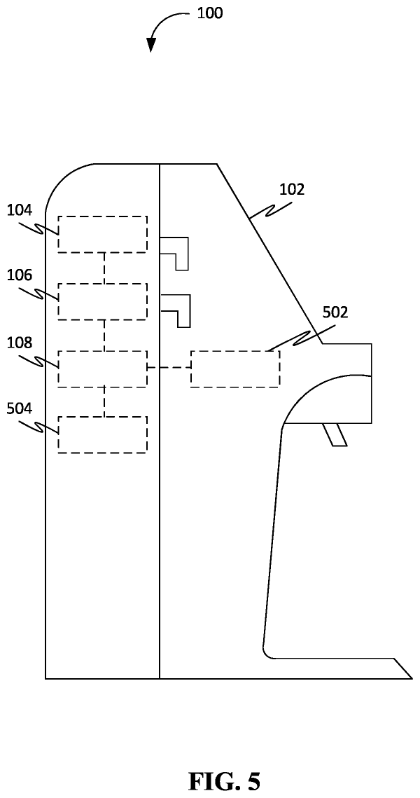

Further, in some embodiments, the processing device 106 may be configured for determining one or more therapeutic environments for the treating of the one or more mental disorders of the one or more users based on the one or more treatment plans. Further, the processing device 106 may be configured for analyzing one or more environment sensor data. Further, the processing device 106 may be configured for generating one or more augmented reality content for the treating of the one or more mental disorders of the one or more users based on the analyzing of the one or more environment sensor data and the determining of the one or more therapeutic environments. Further, the communication device 108 may be configured for receiving the one or more environment sensor data associated with one or more environments of the one or more users from one or more environment sensors 502, as shown in FIG. 5. Further, the one or more environment sensors 502 may be configured for generating the one or more environment sensor data based on detecting one or more a physical characteristic, and a positional characteristic of one or more objects present in the one or more environments of the one or more users. Further, the physical characteristic may include a shape, a color, a texture, etc. Further, the positional characteristic may include a movement, an orientation, etc. Further, the communication device 108 may be configured for transmitting the one or more augmented reality content to one or more augmented reality devices 504, as shown in FIG. 5. Further, the one or more augmented reality devices 504 may be configured for presenting the one or more augmented reality content. Further, the presenting of the one or more augmented reality content creates the one or more therapeutic environments around the one or more users.

Further, in an embodiment, the communication device 108 may be configured for receiving one or more first response data associated with a response of the one or more users to the one or more augmented reality content from the one or more input devices 202. Further, the response may include an involuntary response of the one or more users to the one or more augmented reality content. Further, the involuntary response may include a change in the heartbeat, a change in the level of the one or more hormones, etc. Further, the processing device 106 may be configured for analyzing the one or more first response data. Further, the processing device 106 may be configured for generating a first treatment status of the one or more users based on the analyzing of the one or more first response data. Further, the first treatment status indicates a treatment progress of the one or more users. Further, the generating of the one or more treatment plans may be based on the first treatment status. Further, the storage device 104 may be configured for storing the first treatment status.

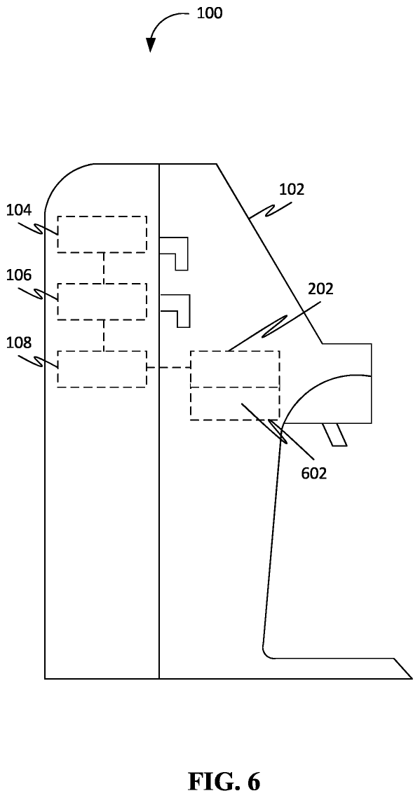

Further, in an embodiment, the one or more input devices 202 may include one or more first sensors 602, as shown in FIG. 6. Further, the one or more first sensors 602 may be configured for generating the one or more first response data based on detecting one or more of a physical parameter, a physiological parameter, and a biological parameter of the one or more users after the presenting of the one or more augmented reality content to the one or more users.

Further, in some embodiments, the one or more treatment plans may include a therapy regime comprising a number of dosages of the one or more types of the one or more psychedelic drugs. Further, the determining of the dosage of the one or more types of the one or more psychedelic drugs may include determining the dosage from the number of dosages of the one or more types of the one or more psychedelic drugs.

Further, in an embodiment, the processing device 106 may be configured for generating a therapy status of the one or more users based on the determining of the dosage from the number of dosages of the one or more types of the one or more psychedelic drugs. Further, the therapy status may indicate a number of completed dosages of the number of dosages administered by the one or more users. Further, the therapy status tracks the dosage administered by the one or more users. Further, the storage device 104 may be configured for storing the therapy status.

Further, in an embodiment, the storage device 104 may be configured for retrieving a historical therapy status of the one or more users. Further, the historical therapy status may indicate a number of previously completed dosages of the number of dosages administered by the one or more users. Further, the processing device 106 may be configured for analyzing the historical therapy status. Further, the determining of the dosage from the number of dosages of the one or more types of the one or more psychedelic drugs may be based on the analyzing of the historical therapy status.

Further, in some embodiments, the communication device 108 may be configured for receiving one or more second response data associated with a response of the one or more users after administering the dosage of the one or more types of the one or more psychedelic drugs by the one or more users from the one or more input devices 202. Further, the response may include an involuntary response of the one or more users towards the one or more types of the one or more psychedelic drugs. Further, the involuntary response may include a change in the heartbeat, a change in the level of the one or more hormones, etc. Further, the processing device 106 may be configured for analyzing the one or more second response data. Further, the processing device 106 may be configured for generating a second treatment status of the one or more users based on the analyzing of the one or more second response data. Further, the first treatment status indicates a treatment progress of the one or more users. Further, the generating of the one or more treatment plans may be based on the second treatment status. Further, the storage device 104 may be configured for storing the second treatment status.

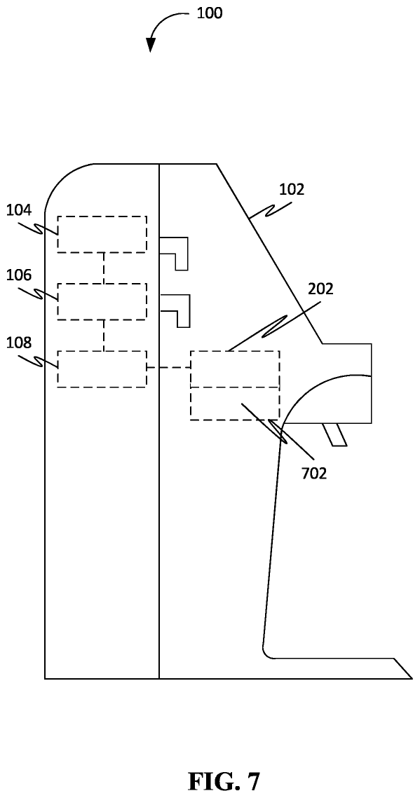

Further, in an embodiment, the one or more input devices 202 may include one or more second sensors 702, as shown in FIG. 7. Further, the one or more second sensors 702 may be configured for generating the one or more second response data based on detecting one or more of a physical parameter, a physiological parameter, and a biological parameter of the one or more users after the administering of the dosage of the one or more types of the one or more drugs by the one or more users.

FIG. 2 is a side view of the apparatus 100, in accordance with some embodiments.

FIG. 3 is a side view of the apparatus 100, in accordance with some embodiments.

FIG. 4 is a side view of the apparatus 100, in accordance with some embodiments.

FIG. 5 is a side view of the apparatus 100, in accordance with some embodiments.

FIG. 6 is a side view of the apparatus 100, in accordance with some embodiments.

FIG. 7 is a side view of the apparatus 100, in accordance with some embodiments.

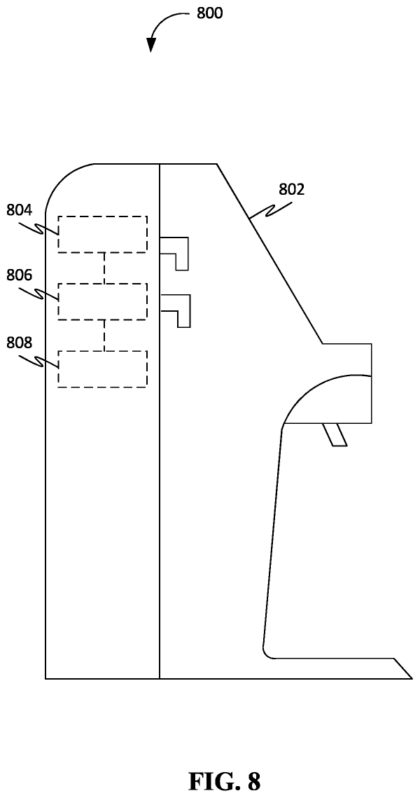

FIG. 8 is a side view of an apparatus 800 for facilitating managing administering of psychedelic drugs to users, in accordance with some embodiments. Further, the apparatus may include a dispenser unit 802, a communication device 808, a processing device 806, and a storage device 804.

Further, the dispenser unit 802 may be configured for dispensing a dosage of one or more types of one or more psychedelic drugs. Further, the one or more types of the one or more psychedelic drugs may be stored in the dispenser unit 802.

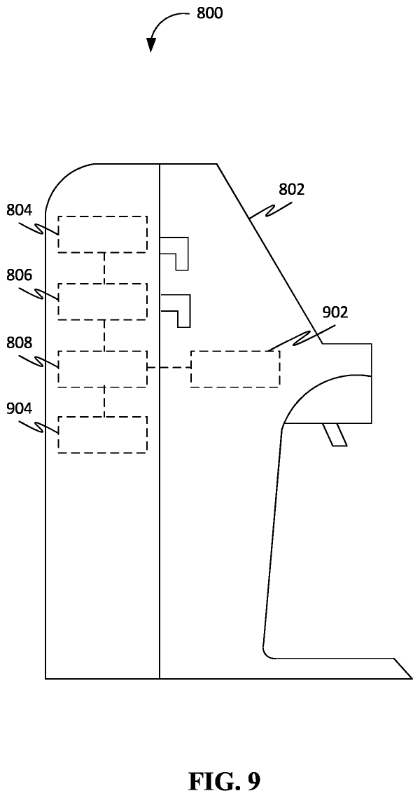

Further, the communication device 808 may be disposed on the dispenser unit 802. Further, the communication device 808 may be configured for receiving one or more user data associated with one or more users from one or more input devices 902, as shown in FIG. 9. Further, the communication device 808 may be configured for transmitting one or more therapist data to one or more output devices 904, as shown in FIG. 9.

Further, the processing device 806 may be disposed on the dispenser unit 802. Further, the processing device 806 may be communicatively coupled with the communication device 808. Further, the processing device 806 may be configured for analyzing the one or more user data. Further, the processing device 806 may be configured for determining one or more mental disorders of the one or more users based on the analyzing of the one or more user data. Further, the processing device 806 may be configured for analyzing one or more clinical data and one or more research data using one or more machine learning models. Further, the processing device 806 may be configured for generating one or more treatment plans for treating the one or more mental disorders of the one or more users based on the analyzing of the one or more user data and the analyzing of the one or more clinical data and the one or more research data. Further, the one or more treatment plans may be personalized to the one or more users. Further, the processing device 806 may be configured for determining the dosage of the one or more types of the one or more psychedelic drugs based on the one or more treatment plans. Further, the processing device 806 may be configured for generating one or more commands for the dispenser unit 802 based on the determining of the dosage of the one or more types of the one or more psychedelic drugs. Further, the dispenser unit 802 may be communicatively coupled with the processing device 806. Further, the dispensing of the dosage of the one or more types of the one or more psychedelic drugs may be based on the one or more commands. Further, the processing device 806 may be configured for identifying one or more therapists for the one or more users based on the one or more treatment plans. Further, the one or more therapists may be personalized to the one or more users.

Further, the storage device 804 may be disposed on the dispenser unit 802. Further, the storage device 804 may be communicatively coupled with the processing device 806. Further, the storage device 804 may be configured for retrieving the one or more clinical data associated with one or more clinical studies on the one or more mental disorders based on the determining of the one or more mental disorders. Further, the storage device 804 may be configured for retrieving the one or more research data associated with a treatment of the one or more mental disorders based on the determining of the one or more mental disorders. Further, the storage device 804 may be configured for storing the one or more machine learning models and the one or more user data. Further, the storage device 804 may be configured for retrieving the one or more therapist data associated with the one or more therapists based on the identifying of the one or more therapists.

Further, in some embodiments, the communication device 808 may be configured for receiving one or more sensor data associated with the one or more users from one or more sensors. Further, the one or more sensors may be configured for generating the one or more sensor data based on detecting one or more of a physical parameter, a physiological parameter, and a biological parameter of the one or more users. Further, the processing device 806 may be configured for analyzing the one or more sensor data. Further, the generating of the one or more treatment plans may be based on the analyzing of the one or more sensor data.

Further, in some embodiments, the processing device 806 may be configured for determining one or more therapeutic environments for the treating of the one or more mental disorders of the one or more users based on the one or more treatment plans. Further, the processing device 806 may be configured for analyzing one or more environment sensor data. Further, the processing device 806 may be configured for generating one or more augmented reality content for the treating of the one or more mental disorders of the one or more users based on the analyzing of the one or more environment sensor data and the determining of the one or more therapeutic environments. Further, the communication device 808 may be configured for receiving the one or more environment sensor data associated with one or more environments of the one or more users from one or more environment sensors. Further, the one or more environment sensors may be configured for generating the one or more environment sensor data based on detecting one or more a physical characteristic, and a positional characteristic of one or more objects present in the one or more environments of the one or more users. Further, the communication device 808 may be configured for transmitting the one or more augmented reality content to one or more augmented reality devices. Further, the one or more augmented reality devices may be configured for presenting the one or more augmented reality content. Further, the presenting of the one or more augmented reality content creates the one or more therapeutic environments around the one or more users

Further, in an embodiment, the communication device 808 may be configured for receiving one or more first response data associated with a response of the one or more users to the one or more augmented reality content from the one or more input devices 902. Further, the processing device 806 may be configured for analyzing the one or more first response data. Further, the processing device 806 may be configured for generating a first treatment status of the one or more users based on the analyzing of the one or more first response data. Further, the generating of the one or more treatment plans may be based on the first treatment status. Further, the storage device 804 may be configured for storing the first treatment status.

Further, in an embodiment, the one or more input devices 902 may include one or more first sensors. Further, the one or more first sensors may be configured for generating the one or more first response data based on detecting one or more of a physical parameter, a physiological parameter, and a biological parameter of the one or more users after the presenting of the one or more augmented reality content to the one or more users.

Further, in some embodiments, the one or more treatment plans may include a therapy regime comprising a number of dosages of the one or more types of the one or more psychedelic drugs. Further, the determining of the dosage of the one or more types of the one or more psychedelic drugs may include determining the dosage from the number of dosages of the one or more types of the one or more psychedelic drugs.

Further, in an embodiment, the processing device 806 may be configured for generating a therapy status of the one or more users based on the determining of the dosage from the number of dosages of the one or more types of the one or more psychedelic drugs. Further, the storage device 804 may be configured for storing the therapy status.

Further, in an embodiment, the storage device 804 may be configured for retrieving a historical therapy status of the one or more users. Further, the processing device 806 may be configured for analyzing the historical therapy status. Further, the determining of the dosage from the number of dosages of the one or more types of the one or more psychedelic drugs may be based on the analyzing of the historical therapy status.

Further, in some embodiments, the communication device 808 may be configured for receiving one or more second response data associated with a response of the one or more users after administering the dosage of the one or more types of the one or more psychedelic drugs by the one or more users from the one or more input devices 902. Further, the processing device 806 may be configured for analyzing the one or more second response data. Further, the processing device 806 may be configured for generating a second treatment status of the one or more users based on the analyzing of the one or more second response data. Further, the generating of the one or more treatment plans may be based on the second treatment status. Further, the storage device 804 may be configured for storing the second treatment status.

FIG. 9 is a side view of the apparatus 800, in accordance with some embodiments.

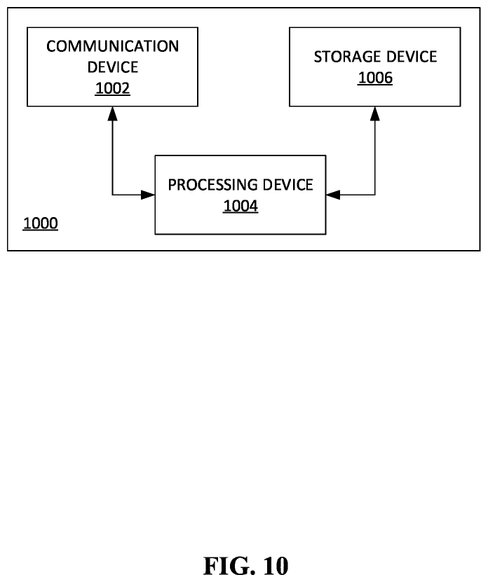

FIG. 10 is a block diagram of a system 1000 for facilitating administering psychedelic dosing and regimen therapy tracking, in accordance with some embodiments. Accordingly, the system 1000 may include a communication device 1002 configured for receiving a request from at least one user device associated with at least one user. Further, the at least one user may include a patient that may be suffering from mental disorders such as opioid addiction, alcohol addiction, smoking addiction, post-traumatic stress disorder, clinical depression, anxiety disorder, dementia, etc. Further, the at least one user device may include a smartphone, a mobile, a tablet, a personal computer, a laptop, and so on. Further, the request may include an indication associated with the at least one user may want to be treated for the mental disorders. Further, the request may include user data. Further, the user data may include personal data and medical data. Further, the personal data may include a user name, contact number, address, email address, etc. Further, the medical data may be associated with at least one of the mental disorders. Further, the medical data include previous medical prescriptions, symptoms, medical examination data, etc. Further, the medical data may facilitate medical screening of the at least one user. Further, the communication device 1002 may be configured for receiving a therapy regime from at least one therapist device. Further, the at least one therapist device may include a smartphone, a mobile, a tablet, a personal computer, a laptop, and so on. Further, the therapy regime may be associated with a therapy that may assist in the treatment of the at least one user. Further, the therapy regime may include a duration of a therapy session and a frequency of the therapy session.

Further, the communication device 1002 may be configured for transmitting the individualized treatment plan to a dispenser. Further, the dispenser may be configured for dispensing the psychedelics based on the individualized treatment plan. Further, the communication device 1002 may be configured for transmitting the individualized treatment plan and the therapy regime to the at least one user device.

Further, the system 1000 may include a processing device 1004 configured for analyzing the request based on at least one artificial learning model. Further, the processing device 1004 may be configured for generating an individualized treatment plan based on the analyzing. Further, the individualized treatment plan may include a dosing regimen associated with psychedelics that may include vapor, liquid, and solid bioactive psychedelic, (alkaloids, polysaccharides, terpenoids, steroids, phenolics) i.e., Psilocybin, PCP, LSD, MDNA, DMT, payote, mescaline, and cannabis. Further, the processing device 1004 may be configured for identifying a trained therapist based on the analyzing and the individualized treatment plan.

Further, the system 1000 may include a storage device 1006 configured for retrieving the at least one artificial intelligence model. Further, the artificial intelligence model may include a machine learning model. Further, the artificial learning model may be based on scientific, medical psychedelic treatment databases with clinical studies, university researched and published formulation, method of delivery, and regimen. Further, the storage device 1006 may be configured for storing the individualized treatment plan and the therapy regime.

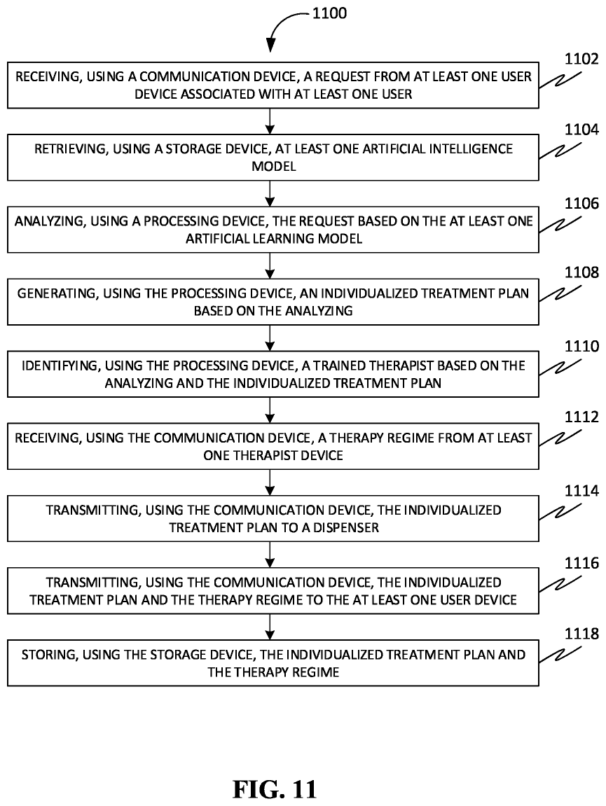

FIG. 11 is a flow chart of a method 1100 for facilitating administering psychedelic dosing and regimen therapy tracking, in accordance with some embodiments. Accordingly, at 1102, the method 1100 may include receiving, using a communication device, a request from at least one user device associated with at least one user. Further, the at least one user may include a patient that may be suffering from mental disorders such as opioid addiction, alcohol addiction, smoking addiction, post-traumatic stress disorder, clinical depression, anxiety disorder, dementia, etc. Further, the at least one user device may include a smartphone, a mobile, a tablet, a personal computer, a laptop, and so on. Further, the request may include an indication associated with the at least one user may want to be treated for the mental disorders. Further, the request may include user data. Further, the user data may include personal data and medical data. Further, the personal data may include a user name, a contact number, a address, an email address, etc. Further, the medical data may be associated with at least one of the mental disorders. Further, the medical data include previous medical prescriptions, symptoms, medical examination data, etc. Further, the medical data may facilitate medical screening of the at least one user.

Further, at 1104, the method 1100 may include retrieving, using a storage device, at least one artificial intelligence model. Further, the artificial intelligence model may include a machine learning model. Further, the artificial learning model may be based on scientific, medical psychedelic treatment databases with clinical studies, university researched and published formulation, method 1100 of delivery, and regimen.

Further, at 1106, the method 1100 may include analyzing, using a processing device, the request based on the at least one artificial learning model.

Further, at 1108, the method 1100 may include generating, using the processing device, an individualized treatment plan based on the analyzing. Further, the individualized treatment plan may include a dosing regimen associated with psychedelics that may include vapor, liquid, and solid bioactive psychedelic, (alkaloids, polysaccharides, terpenoids, steroids, phenolics) i.e., Psilocybin, PCP, LSD, MDNA, DMT, payote, mescaline, and cannabis

Further, at 1110, the method 1100 may include identifying, using the processing device, a trained therapist based on the analyzing and the individualized treatment plan.

Further, at 1112, the method 1100 may include receiving, using the communication device, a therapy regime from at least one therapist device. Further, the at least one therapist device may include a smartphone, a mobile, a tablet, a personal computer, a laptop, and so on. Further, the therapy regime may be associated with a therapy that may assist in the treatment of the at least one user. Further, the therapy regime may include a duration of a therapy session and a frequency of the therapy session.

Further, at 1114, the method 1100 may include transmitting, using the communication device, the individualized treatment plan to a dispenser (or at home apparatus dispenser/apparatus 1318). Further, the dispenser may be configured for facilitating administering psychedelic dosing based on the individualized treatment plan.

Further, at 1116, the method 1100 may include transmitting, using the communication device, the individualized treatment plan and the therapy regime to the at least one user device.

Further, at 1118, the method 1100 may include storing, using the storage device, the individualized treatment plan and the therapy regime.

Further, in some embodiments, the method 1100 may include receiving, using the communication device, tracking data associated with dispensing of the psychedelics from the dispenser. Further, the tracking data may be associated with the psychedelics. Further, the tracking data may include a quantity of the psychedelics dispensed and a frequency of the psychedelics being dispensed (or dispersed). Further, the method 1100 may include processing, using the processing device, the tracking data. Further, the method 1100 may include modifying, using the processing device, the individualized treatment plan based on the processing to generate a modified treatment plan. Further, the method 1100 may include transmitting, using the communication device, the modified treatment plan to the at least one user device. Further, the method 1100 may include transmitting, using the communication device, the modified treatment plan to the dispenser. Further, the administering of the psychedelics may be based on the modified treatment plan.

Further, in some embodiments, the method 1100 may include receiving, using the communication device, treatment progress data from the at least one user device. Further, the treatment progress data may facilitate tracking treatment of the at least one user based on the individualized treatment plan. Further, the treatment progress data may be associated with a medical condition of the at least one user suffering from at least one of the mental disorders. Further, the treatment progress data may include medical symptoms, medical examination reports, physiological reports, etc. associated with the at least one user. Further, the method 1100 may include analyzing, using the processing device, at least one of the tracking data, the medical data, the treatment progress data, and the therapy regime to generate a progress report. Further, the method 1100 may include transmitting, using the communication device, the progress report to at least one of the at least one user device and the at least one therapist device. Further, the generating of the modified treatment plan may be based on the analyzing of at least one of the tracking data, the medical data, the treatment progress data, and the therapy regime.

Further, in some embodiments, the method 1100 may include receiving, using the communication device, a modified therapy regimen based on the transmitting of the progress report. Further, the method 1100 may include transmitting, using the communication device, the progress report to the at least one user device.

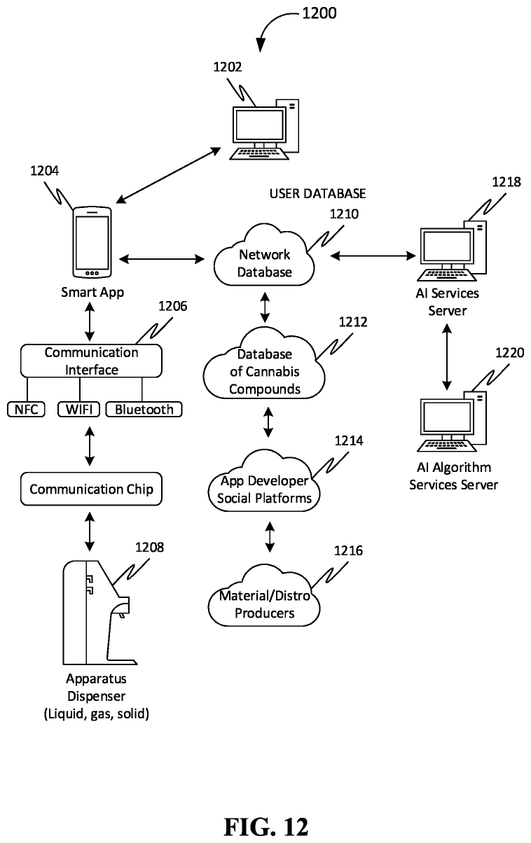

FIG. 12 is a schematic of a system 1200 for facilitating administering psychedelic dosing and regimen therapy tracking, in accordance with some embodiments. Accordingly, the system 1200 may include a user database 1202 associated with a software application (or smartphone app) that may be compiled on a smartphone 1204. Further, the smartphone 1204 may include a communication interface 1206. Further, the smartphone 1204 may be communicatively coupled to an apparatus dispenser 1208 (such as the apparatus 1318). Further, the apparatus dispenser 1208 may dispense liquid, solid, and gas psychedelics. Further, the smartphone 1204 may be associated with a network database 1210. Further, the network database 1210 may include a database of cannabis compounds 1212, app developer social platform 1214, and material producers/distributors 1216. Further, the system 1200 may include an AI services server 1218 and an AI algorithm services server 1220 associated with the network database 1210.

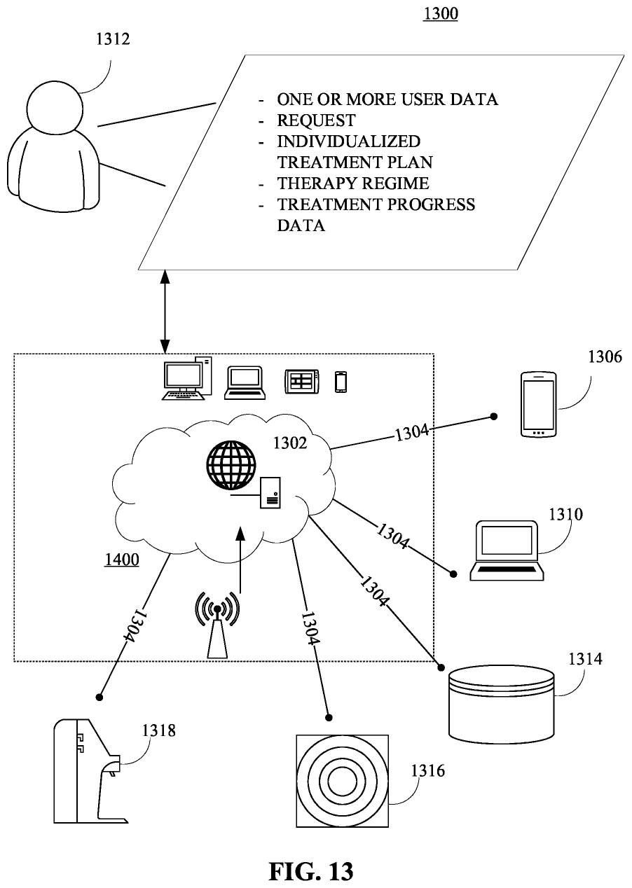

FIG. 13 is an illustration of an online platform 1300 consistent with various embodiments of the present disclosure. By way of non-limiting example, the online platform 1300 to facilitate managing administering of psychedelic drugs to users may be hosted on a centralized server 1302, such as, for example, a cloud computing service. The centralized server 1302 may communicate with other network entities, such as, for example, a mobile device 1306 (such as a smartphone, a laptop, a tablet computer etc.), other electronic devices 1310 (such as desktop computers, server computers etc.), databases 1314, sensors 1316, and an apparatus 1318 (such as the apparatus 100, the apparatus 800, etc.) over a communication network 1304, such as, but not limited to, the Internet. Further, users of the online platform 1300 may include relevant parties such as, but not limited to, end-users, administrators, service providers, service consumers and so on. Accordingly, in some instances, electronic devices operated by the one or more relevant parties may be in communication with the platform.

A user 1312, such as the one or more relevant parties, may access online platform 1300 through a web based software application or browser. The web based software application may be embodied as, for example, but not be limited to, a website, a web application, a desktop application, and a mobile application compatible with a computing device 1400.

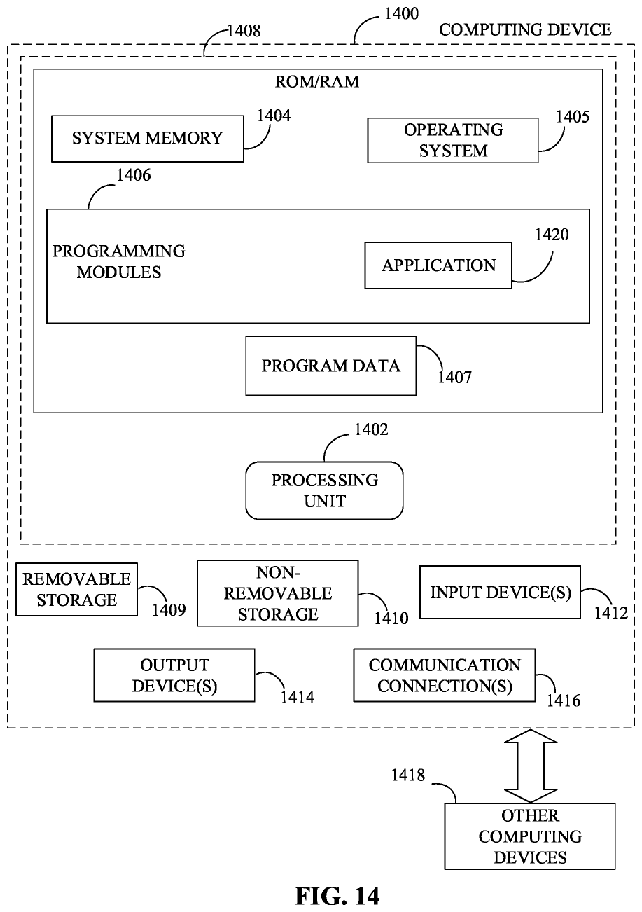

With reference to FIG. 14, a system consistent with an embodiment of the disclosure may include a computing device or cloud service, such as computing device 1400. In a basic configuration, computing device 1400 may include at least one processing unit 1402 and a system memory 1404. Depending on the configuration and type of computing device, system memory 1404 may comprise, but is not limited to, volatile (e.g. random-access memory (RAM)), non-volatile (e.g. read-only memory (ROM)), flash memory, or any combination. System memory 1404 may include operating system 1405, one or more programming modules 1406, and may include a program data 1407. Operating system 1405, for example, may be suitable for controlling computing device 1400's operation. In one embodiment, programming modules 1406 may include image-processing module, machine learning module. Furthermore, embodiments of the disclosure may be practiced in conjunction with a graphics library, other operating systems, or any other application program and is not limited to any particular application or system. This basic configuration is illustrated in FIG. 14 by those components within a dashed line 1408.

Computing device 1400 may have additional features or functionality. For example, computing device 1400 may also include additional data storage devices (removable and/or non-removable) such as, for example, magnetic disks, optical disks, or tape. Such additional storage is illustrated in FIG. 14 by a removable storage 1409 and a non-removable storage 1410. Computer storage media may include volatile and non-volatile, removable and non-removable media implemented in any method or technology for storage of information, such as computer-readable instructions, data structures, program modules, or other data. System memory 1404, removable storage 1409, and non-removable storage 1410 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 1400. Any such computer storage media may be part of device 1400. Computing device 1400 may also have input device(s) 1412 such as a keyboard, a mouse, a pen, a sound input device, a touch input device, a location sensor, a camera, a biometric sensor, etc. Output device(s) 1414 such as a display, speakers, a printer, etc. may also be included. The aforementioned devices are examples and others may be used.

Computing device 1400 may also contain a communication connection 1416 that may allow device 1400 to communicate with other computing devices 1418, such as over a network in a distributed computing environment, for example, an intranet or the Internet. Communication connection 1416 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 1404, including operating system 1405. While executing on processing unit 1402, programming modules 1406 (e.g., application 1420 such as a media player) may perform processes including, for example, one or more stages of methods, algorithms, systems, applications, servers, databases as described above. The aforementioned process is an example, and processing unit 1402 may perform other processes. Other programming modules that may be used in accordance with embodiments of the present disclosure may include machine learning applications.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general purpose graphics processor-based systems, multiprocessor systems, microprocessor-based or programmable consumer electronics, application specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

Although the present disclosure has been explained in relation to its preferred embodiment, it is to be understood that many other possible modifications and variations can be made without departing from the spirit and scope of the disclosure.

What is claimed is:

1. An apparatus for facilitating managing administering of psychedelic drugs to users, the apparatus comprising:
a dispenser unit configured for dispensing a dosage of one or more types of one or more psychedelic drugs, wherein the one or more types of the one or more psychedelic drugs are stored in the dispenser unit;
a communication device disposed on the dispenser unit, wherein the communication device is configured for receiving one or more user data associated with one or more users from one or more input devices;
a processing device disposed on the dispenser unit, wherein the processing device is communicatively coupled with the communication device, wherein the processing device is configured for:
analyzing the one or more user data;
determining one or more mental disorders of the one or more users based on the analyzing of the one or more user data;
analyzing one or more clinical data and one or more research data using one or more machine learning models;
generating one or more treatment plans for treating the one or more mental disorders of the one or more users based on the analyzing of the one or more user data and the analyzing of the one or more clinical data and the one or more research data, wherein the one or more treatment plans are personalized to the one or more users;
determining the dosage of the one or more types of the one or more psychedelic drugs based on the one or more treatment plans; and
generating one or more commands for the dispenser unit based on the determining of the dosage of the one or more types of the one or more psychedelic drugs, wherein the dispenser unit is communicatively coupled with the processing device, wherein the dispensing of the dosage of the one or more types of the one or more psychedelic drugs is based on the one or more commands; and
a storage device disposed on the dispenser unit, wherein the storage device is communicatively coupled with the processing device, wherein the storage device is configured for:
retrieving the one or more clinical data associated with one or more clinical studies on the one or more mental disorders based on the determining of the one or more mental disorders;
retrieving the one or more research data associated with a treatment of the one or more mental disorders based on the determining of the one or more mental disorders; and
storing the one or more machine learning models and the one or more user data.

2. The apparatus of claim 1, wherein the processing device is further configured for identifying one or more therapists for the one or more users based on the one or more treatment plans, wherein the one or more therapists are personalized to the one or more users, wherein the storage device is further configured for retrieving one or more therapist data associated with the one or more therapists based on the identifying of the one or more therapists, wherein the communication device is further configured for transmitting the one or more therapist data to one or more output devices.

3. The apparatus of claim 1, wherein the communication device is further configured for receiving one or more sensor data associated with the one or more users from one or more sensors, wherein the one or more sensors is configured for generating the one or more sensor data based on detecting one or more of a physical parameter, a physiological parameter, and a biological parameter of the one or more users, wherein the processing device is further configured for analyzing the one or more sensor data, wherein the generating of the one or more treatment plans is further based on the analyzing of the one or more sensor data.

4. The apparatus of claim 1, wherein the processing device is further configured for:
determining one or more therapeutic environments for the treating of the one or more mental disorders of the one or more users based on the one or more treatment plans;
analyzing one or more environment sensor data; and
generating one or more augmented reality content for the treating of the one or more mental disorders of the one or more users based on the analyzing of the one or more environment sensor data and the determining of the one or more therapeutic environments, wherein the communication device is further configured for:
receiving the one or more environment sensor data associated with one or more environments of the one or more users from one or more environment sensors, wherein the one or more environment sensors is configured for generating the one or more environment sensor data based on detecting one or more a physical characteristic, and a positional characteristic of one or more objects present in the one or more environments of the one or more users; and
transmitting the one or more augmented reality content to one or more augmented reality devices, wherein the one or more augmented reality devices is configured for presenting the one or more augmented reality content, wherein the presenting of the one or more augmented reality content creates the one or more therapeutic environments around the one or more users.

5. The apparatus of claim 4, wherein the communication device is further configured for receiving one or more first response data associated with a response of the one or more users to the one or more augmented reality content from the one or more input devices, wherein the processing device is further configured for:
analyzing the one or more first response data; and
generating a first treatment status of the one or more users based on the analyzing of the one or more first response data, wherein the generating of the one or more treatment plans is further based on the first treatment status, wherein the storage device is further configured for storing the first treatment status.

6. The apparatus of claim 5, wherein the one or more input devices comprises one or more first sensors, wherein the one or more first sensors is configured for generating the one or more first response data based on detecting one or more of a physical parameter, a physiological parameter, and a biological parameter of the one or more users after the presenting of the one or more augmented reality content to the one or more users.

7. The apparatus of claim 1, wherein the one or more treatment plans comprises a therapy regime comprising a number of dosages of the one or more types of the one or more psychedelic drugs, wherein the determining of the dosage of the one or more types of the one or more psychedelic drugs comprises determining the dosage from the number of dosages of the one or more types of the one or more psychedelic drugs.

8. The apparatus of claim 7, wherein the processing device is further configured for generating a therapy status of the one or more users based on the determining of the dosage from the number of dosages of the one or more types of the one or more psychedelic drugs, wherein the storage device is configured for storing the therapy status.

9. The apparatus of claim 7, wherein the storage device is further configured for retrieving a historical therapy status of the one or more users, wherein the processing device is further configured for analyzing the historical therapy status, wherein the determining of the dosage from the number of dosages of the one or more types of the one or more psychedelic drugs is further based on the analyzing of the historical therapy status.

10. The apparatus of claim 1, wherein the communication device is further configured for receiving one or more second response data associated with a response of the one or more users after administering the dosage of the one or more types of the one or more psychedelic drugs by the one or more users from the one or more input devices, wherein the processing device is further configured for:
analyzing the one or more second response data; and
generating a second treatment status of the one or more users based on the analyzing of the one or more second response data, wherein the generating of the one or more treatment plans is further based on the second treatment status, wherein the storage device is further configured for storing the second treatment status.

11. The apparatus of claim 9, wherein the one or more input devices comprises one or more second sensors, wherein the one or more second sensors is configured for generating the one or more second response data based on detecting one or more of a physical parameter, a physiological parameter, and a biological parameter of the one or more users after the administering of the dosage of the one or more types of the one or more drugs by the one or more users.

12. An apparatus for facilitating managing administering of psychedelic drugs to users, the apparatus comprising:
a dispenser unit configured for dispensing a dosage of one or more types of one or more psychedelic drugs, wherein the one or more types of the one or more psychedelic drugs are stored in the dispenser unit;
a communication device disposed on the dispenser unit, wherein the communication device is configured for:
receiving one or more user data associated with one or more users from one or more input devices; and
transmitting one or more therapist data to one or more output devices;
a processing device disposed on the dispenser unit, wherein the processing device is communicatively coupled with the communication device, wherein the processing device is configured for:
analyzing the one or more user data;
determining one or more mental disorders of the one or more users based on the analyzing of the one or more user data;
analyzing one or more clinical data and one or more research data using one or more machine learning models;
generating one or more treatment plans for treating the one or more mental disorders of the one or more users based on the analyzing of the one or more user data and the analyzing of the one or more clinical data and the one or more research data, wherein the one or more treatment plans are personalized to the one or more users;
determining the dosage of the one or more types of the one or more psychedelic drugs based on the one or more treatment plans;
generating one or more commands for the dispenser unit based on the determining of the dosage of the one or more types of the one or more psychedelic drugs, wherein the dispenser unit is communicatively coupled with the processing device, wherein the dispensing of the dosage of the one or more types of the one or more psychedelic drugs is based on the one or more commands; and
identifying one or more therapists for the one or more users based on the one or more treatment plans, wherein the one or more therapists are personalized to the one or more users; and
a storage device disposed on the dispenser unit, wherein the storage device is communicatively coupled with the processing device, wherein the storage device is configured for:
retrieving the one or more clinical data associated with one or more clinical studies on the one or more mental disorders based on the determining of the one or more mental disorders;
retrieving the one or more research data associated with a treatment of the one or more mental disorders based on the determining of the one or more mental disorders;
storing the one or more machine learning models and the one or more user data; and
retrieving the one or more therapist data associated with the one or more therapists based on the identifying of the one or more therapists.

13. The apparatus of claim 12, wherein the communication device is further configured for receiving one or more sensor data associated with the one or more users from one or more sensors, wherein the one or more sensors is configured for generating the one or more sensor data based on detecting one or more of a physical parameter, a physiological parameter, and a biological parameter of the one or more users, wherein the processing device is further configured for analyzing the one or more sensor data, wherein the generating of the one or more treatment plans is further based on the analyzing of the one or more sensor data.

14. The apparatus of claim 12, wherein the processing device is further configured for:
determining one or more therapeutic environments for the treating of the one or more mental disorders of the one or more users based on the one or more treatment plans;
analyzing one or more environment sensor data; and
generating one or more augmented reality content for the treating of the one or more mental disorders of the one or more users based on the analyzing of the one or more environment sensor data and the determining of the one or more therapeutic environments, wherein the communication device is further configured for:
receiving the one or more environment sensor data associated with one or more environments of the one or more users from one or more environment sensors, wherein the one or more environment sensors is configured for generating the one or more environment sensor data based on detecting one or more a physical characteristic, and a positional characteristic of one or more objects present in the one or more environments of the one or more users; and
transmitting the one or more augmented reality content to one or more augmented reality devices, wherein the one or more augmented reality devices is configured for presenting the one or more augmented reality content, wherein the presenting of the one or more augmented reality content creates the one or more therapeutic environments around the one or more users.

15. The apparatus of claim 14, wherein the communication device is further configured for receiving one or more first response data associated with a response of the one or more users to the one or more augmented reality content from the one or more input devices, wherein the processing device is further configured for:
analyzing the one or more first response data; and
generating a first treatment status of the one or more users based on the analyzing of the one or more first response data, wherein the generating of the one or more treatment plans is further based on the first treatment status, wherein the storage device is further configured for storing the first treatment status.

16. The apparatus of claim 15, wherein the one or more input devices comprises one or more first sensors, wherein the one or more first sensors is configured for generating the one or more first response data based on detecting one or more of a physical parameter, a physiological parameter, and a biological parameter of the one or more users after the presenting of the one or more augmented reality content to the one or more users.

17. The apparatus of claim 12, wherein the one or more treatment plans comprises a therapy regime comprising a number of dosages of the one or more types of the one or more psychedelic drugs, wherein the determining of the dosage of the one or more types of the one or more psychedelic drugs comprises determining the dosage from the number of dosages of the one or more types of the one or more psychedelic drugs.

18. The apparatus of claim 17, wherein the processing device is further configured for generating a therapy status of the one or more users based on the determining of the dosage from the number of dosages of the one or more types of the one or more psychedelic drugs, wherein the storage device is configured for storing the therapy status.

19. The apparatus of claim 17, wherein the storage device is further configured for retrieving a historical therapy status of the one or more users, wherein the processing device is further configured for analyzing the historical therapy status, wherein the determining of the dosage from the number of dosages of the one or more types of the one or more psychedelic drugs is further based on the analyzing of the historical therapy status.

20. The apparatus of claim 12, wherein the communication device is further configured for receiving one or more second response data associated with a response of the one or more users after administering the dosage of the one or more types of the one or more psychedelic drugs by the one or more users from the one or more input devices, wherein the processing device is further configured for:
analyzing the one or more second response data; and
generating a second treatment status of the one or more users based on the analyzing of the one or more second response data, wherein the generating of the one or more treatment plans is further based on the second treatment status, wherein the storage device is further configured for storing the second treatment status.
